(19) United States
(12) Patent Application Publication
(10) Pub. No.: US 2007/0112329 A1
(43) Pub. Date: May 17, 2007

(54) Infiltration detection system

(76) Inventor: Burton H. Sage JR., Carlsbad, CA (US)

Correspondence Address: BURTON H. SAGE, JR, 2453 IMPALA DRIVE, CARLSBAD, CA, 92010, US

(21) Appl. No.: 11/593,674

(22) Filed: Nov. 7, 2006

Related U.S. Application Data
(60) Provisional application No. 60/734,473, filed on Nov. 8, 2005.

(51) Int. Cl. A61M 31/00

(52) U.S. Cl. 604/507; 604/257

(57) ABSTRACT

An IV infusion system capable of detecting infiltration/extravasation events is described. Flow rate changes due to venous pressure variations are measured; infiltration/extravasation events are detected when these flow rate patterns are altered when infusion is not into a vein.

This application claims priority to and subject matter disclosed in provisional application No. 60/734,473, filed on Nov. 8, 2005; the content of this application being incorporated by reference herein in its entirety.

### FIELD OF THE INVENTION

This application relates to the delivery of pharmaceutical solutions to the body of an animal and more specifically to systems and methods capable of detecting when the delivery of these solutions has resulted in an infiltration or extravasation event.

### BACKGROUND AND RELATED ART

The intravenous delivery of pharmaceutical solutions to patients, especially in a hospital and other settings where professional medical attention is available has many advantages. First, relatively large volumes of solutions may be administered without pain and discomfort due to distention of tissue. Second, putting solutions into the blood stream insures rapid distribution to the rest of the body such that it quickly reaches the desired site of action. Third, when the solutions reach the blood stream a rapid dilution of the solution occurs. This rapid dilution allows highly toxic solutions such as vesicants and other oncological agents to be safely and effectively delivered. And fourth, once an IV line is properly set, other pharmaceutical solutions may be administered using the IV line without causing the patient the pain and discomfort of additional injections. For these and other reasons, intravenous administration of pharmaceutical solutions has become a standard of care and it is now quite unusual for a patient in the hospital to not have pharmaceutical solutions administered this way.

For many decades prior to the latter part of the 20^{th} century, these pharmaceutical solutions were administered to the patient by means of a gravity bag or bottle. In such a system, the gravity bag or bottle is placed on a support above the patient. An IV line is run from the bag or bottle to the patient so that the pressure head generated by having the solution above the patient provides the motive force for moving the solution from the bag into the patient. The flow rate of the solution into the patient is usually adjusted using a roller clamp which acts as a flow rate valve. In these gravity delivery systems, the fluid driving pressure is quite low, on the order of 1 pound per square inch.

This pressure provides more than adequate flow of the solution when the exit port of the IV line is properly located in the vein. When the exit port is not in the vein, fluid “infiltrates” the surrounding tissue. In the case of infiltration, very little of the solution actually reaches the vein. Infiltration causes two problems. First, the benefits of the pharmaceutical solution are not realized since the solution does not reach the desired sites of action. And second, for certain solutions such as vesicants and other toxic agents, the high concentration of the undiluted solution can cause damage to the tissue.

In a gravity bag infusion system the flow rate is dramatically reduced when an infiltration occurs since the body tissue where the exit port then resides is not capable of receiving the solution at the same rate as the vein. The resulting back pressure limits the amount of fluid reaching the tissue thereby limiting tissue damage.

However, gravity delivery systems have limited accuracy due to difficulty in accurately setting the flow rate initially and in frequently occurring changes in the flow rate due to back pressures from the body such as partial line occlusions, collapsing veins and kinks in the IV line which might result from patient movement. Since the safety and effectiveness of pharmaceutical solutions is based on having the correct concentration of the active agent in the blood stream, achieving the desired active agent concentration with gravity based delivery systems is difficult. In the latter part of the 20^{th} century, more accurate volumetric infusion pumps became the standard of care for IV delivery of pharmaceutical solutions. These volumetric infusion pumps were able to provide accurate delivery since the initial flow rate could be very accurately set and the pump could provided sufficient delivery pressure to overcome any changes in back pressure.

Unfortunately, in the case where the exit port of the IV line is not in the vein, the naturally occurring change in back pressure, which limits the amount of solution which could infiltration the body in a gravity system, is easily overcome by the infusion pump. The result is large amounts of the pharmaceutical solution being delivered to tissue outside the vein. In the case where the solution is toxic, significant tissue damage can result. In exceptional cases, limbs must be amputated because of the amount of damage.

Attempts to detect infiltration during IV administration depend upon trying to detect changes at the infusion site due to the increase in the volume of the solution in the tissue. Lichtenstein in U.S. Pat. No. 4,378,808 employed liquid crystals in contact with the infusion site in an attempt to measure a change in tissue temperature. Since the solution is at room temperature, a significant volume of the solution in the tissue would lower the temperature of the infusion site. The liquid crystals would change color, thus detecting an infiltration. Unfortunately, a large volume of infiltrated fluid in the tissue is needed to detect an infiltration this way. Tissue damage may already be done before the infiltration is detected. And if the infusion rate is low, the tissue will absorb the fluid before a temperature change is detected. Nelson in U.S. Pat. No. 4,534,756 adapted pressure sensors to the IV infusion line to detect the change in back pressure that would result during an infiltration. Unfortunately, back pressure in the infusion line can result from many situations other than infiltration. The result of using back pressure to detect infiltration was a very high number of false alarms. Atkins, et. al. in U.S. Pat. No. 4,877,034 employed an optical sensor in an attempt to detect infiltration. By monitoring several different optical wavelengths of radiation issuing from the infusion site, changes resulting from addition of fluid to the tissue surrounding the infusion site could be detected. Some of these intensity changes could result from tissue temperature change, some could result from the addition of the solution, and some could result from dilution of tissue compounds. Again, large amounts of fluid must infiltrate the infusion site before any infiltration is detected.

Today there are no commercial systems in wide use for the early detection of infiltration of IV solutions into tissue. With the widespread use of IV infusion pumps, tissue extravasation resulting from infiltration of solution from an IV line is a major source of litigation. Thus there is a need for improved methods of early detection of tissue infiltration of IV solutions.

### SUMMARY OF THE INVENTION

Pressure waves due to the beating of the heart are obviously present in the arteries of the body. Similar pressure waves are also present in the veins of the body. An excellent discussion of such waves is given by Jonathan B. Mark, MD in “Getting the most from a CVP Catheter” at the 53^{rd} Annual Refresher Course Lectures presented by the American Society of Anesthesiologists, Oct. 16-20, 2002 at the Orange County Convention Center, the contents of which are incorporated herein in their entirely by reference. These venous pressure waves are of reduced amplitude compared to the pressure waves in the arteries, making their detection more problematic. However, in a fluid delivery system wherein the fluid driving pressure is essentially constant and where back pressures are relatively stable for time periods of seconds up to minutes, these pressure waves cause measurable changes in the fluid delivery rate. It is an objective of this invention to provide a flow sensor in an IV infusion system to measure changes in flow as a result of the normally present venous pressure changes. The flow rate sensor may measure volumetric flow rate, the velocity of the flow stream at one or more locations in the flow tube, or the overall average flow stream velocity, or pressure changes across a fixed flow resistor, or any other flow parameter capable of providing a prominent display of changes in the flow due to venous pressure fluctuations. These venous pressure induced flow changes will be reduced in amplitude or absent when IV delivery is to tissue instead of the vein. When these flow rates changes are measured and displayed, they provide a clear indication of where in the body, that is tissue vs. vein, the fluid is actually being delivered. These flow rate changes can be used as an indication of proper placement of the exit port of an IV infusion set. For the purposes of this invention, a properly placed exit port will be one that is in the vein such that the venous pressure induced flow rate changes are prominent and easily discernable. An improperly placed exit port is one such that the exit port is not in the vein or is against the wall of a vein or has been partially occluded by a clot or bacterial growth or other obstruction such that the pressure induced flow rate changes are less prominent or absent.

It is a further object of the invention to provide a real time display of the flow rate for the medical professional who initially places the exit port of the IV infusion set in the vein of the subject. This medical professional or any medical profession providing care to the patient receiving the IV infusion can observe this display, which shows the amplitude of the flow rate signal, and adjust the placement of the exit port to its proper location in the vein. Prominent signals in terms of amplitude of the flow rate changes will indicate proper placement, weak or absent signals will indicate improper placement.

It is yet another object of the invention to provide a processor for the flow rate signals such that automatic and rapid detection of movement of the exit port from a proper position in the vein to an improper position outside a vein might be made. The processor can further provide a signal to a medical professional responsible for management of the IV infusion that patency of the exit port in the vein has been lost and that an infiltration is likely in process.

Another object of the invention is to provide a method whereby proper placement of the exit port of an IV infusion set in the vein may be accomplished. This method includes providing information to the medical professional regarding flow rate changes due to venous pressure fluctuations. Using a display of the information, and the relative prominence of the displayed flow rate changes, the medical professional can adjust the position of the exit port of the IV infusion set in the vein to optimize prominence of the flow rate changes, thereby properly placing the exit port in the vein.

Yet another object of the invention is to provide a method of reducing the amount of fluid reaching the extravascular space thereby reducing the amount of tissue damage in the event the infusion solution is toxic. This objective is achieved by both providing information to permit proper placement of the exit port when the infusion is started and by alerting the medical professional in the event the exit port moves to an extravascular location at any time during the infusion.

### BRIEF DESCRIPTION OF THE FIGURES

FIG. 1 is an illustration of a gravity infusion system with a flow monitor

FIG. 2 is a schematic of the sensor based flow monitor

FIG. 3 is an electrical equivalent circuit of the IV infusion system of the invention.

FIG. 4 is an illustration of normal venous pressure waveforms.

FIG. 5 is an illustration of attenuation of venous pressure waveforms when the exit port of an IV infusion system moves to an extravascular location.

FIG. 6 shows data comparing the measured time of flight of constant flow and of a sinusoidal pressure wave less than published venous pressure waves.

FIG. 7 shows additional data comparing the measured time of flight of constant flow and of a sinusoidal pressure wave less than published venous pressure waves.

### DETAILED DESCRIPTION OF THE INVENTION

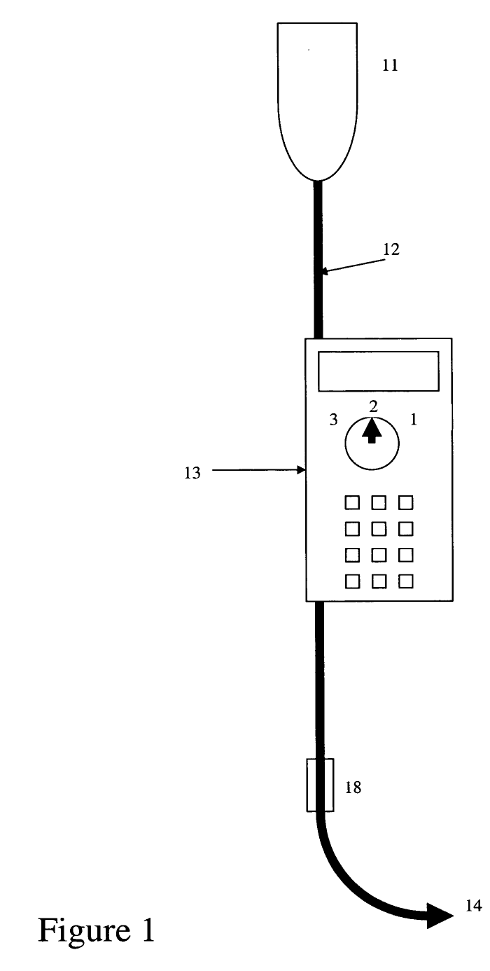

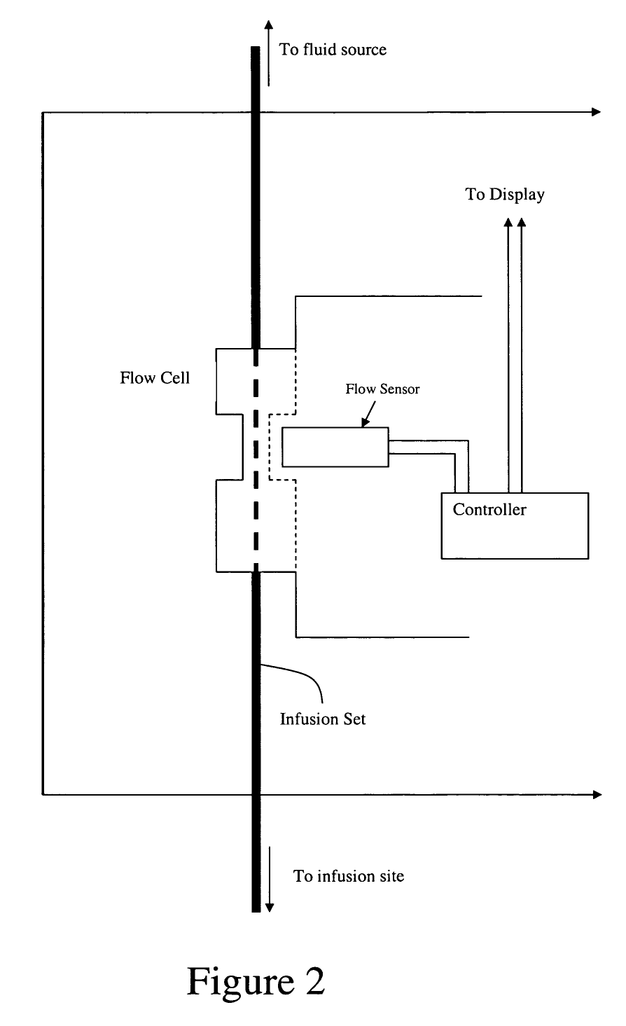

FIG. 1 shows an IV infusion system with a flow monitor capable of measuring the flow of an infusion liquid into a body. Pharmaceutical solution reservoir 11 is connected to IV infusion set 12. Flow monitor 13 is mated to IV infusion set 12. IV infusion set 12 also comprises roller clamp 18 before it passes to the body at position 14. Pharmaceutical solution container 11 can be a glass bottle or plastic bag or syringe with a vented spike or any other suitable container as is known in the art. The volumes of such containers may be as small as 10 milliliters up to a liter. Infusion set 12 can be any IV infusion set provided it comprises a region where the flow is measured as is shown in FIG. 2. The flow cell shown in FIG. 2 can be added to any IV line by Luer connectors, solvent welding or one of a number of different methods as is known in the art. IV infusion set 12 has an exit port 14 which is typically a cannula (not shown) for body access. Flow monitor 13 serves one or more functions including regulating flow, measuring flow, displaying drug infusion data, and displaying flow measurement data. A numerical keyboard is included for a user interface so that a user may select or alter desired drug infusion parameters.

FIG. 2 is a schematic of the flow measurement portion of flow monitor 13. As is shown, when infusion set 12 is mated to flow monitor 13, the flow cell mates with the flow sensor subsystem of flow monitor 13. The flow sensor is capable of measuring properties of the liquid stream during infusion of the pharmaceutical solution. The measurable properties include, but are not limited to volumetric flow rate, velocity of the liquid stream at various locations in the flow cell, average stream velocity, and pressure differences across the flow cell. The flow sensor may take one of several forms. For example, the flow sensor may be an optical flow sensor as taught by Sage in U.S. Pat. No. 6,582,393. Or, the flow sensor may be a thermal time of flight sensor as developed and manufactured by Sensirian, Inc. (www.sensirian.com). Alternatively the flow sensor may be an optical interference sensor as taught by Yin, et al in U.S. Pat. No. 6,386,050. Or, the flow sensor may be a Coriolis Effect based sensor as taught by Najafi et al in US patent publication 20030061889. The sensor may also comprise pressure sensors that measure the pressure difference between the inlet and outlet of the flow cell as shown in FIG. 2. This pressure difference can be used to calculate other flow properties provided other information such as temperature and fluid viscosity are known.

The flow sensor is electrically connected to a processor to manipulate the flow sensor signals to provide flow information which may then be displayed. Such flow information includes, but is not limited to a graphical display of the flow rate over a period of time, the current instantaneous flow rate, the volume of fluid delivered since the infusion started, the volume of fluid remaining to be delivered (assuming the user entered the volume of fluid in the reservoir, and others as may be important to users.

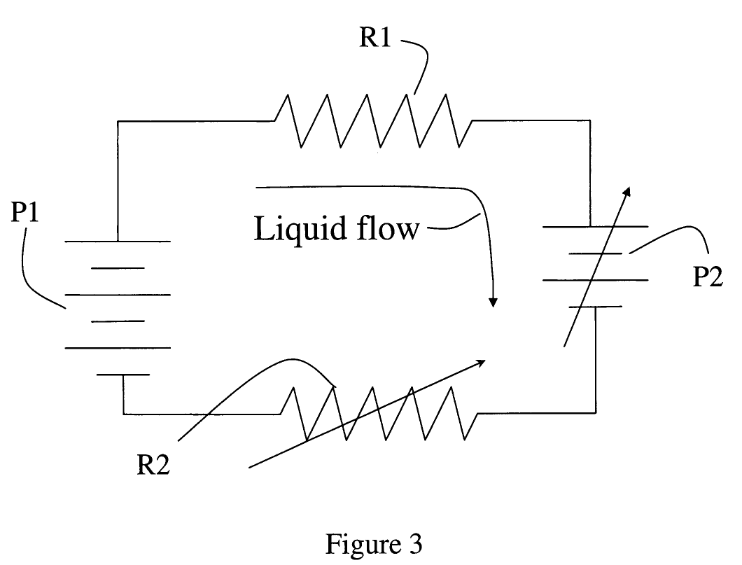

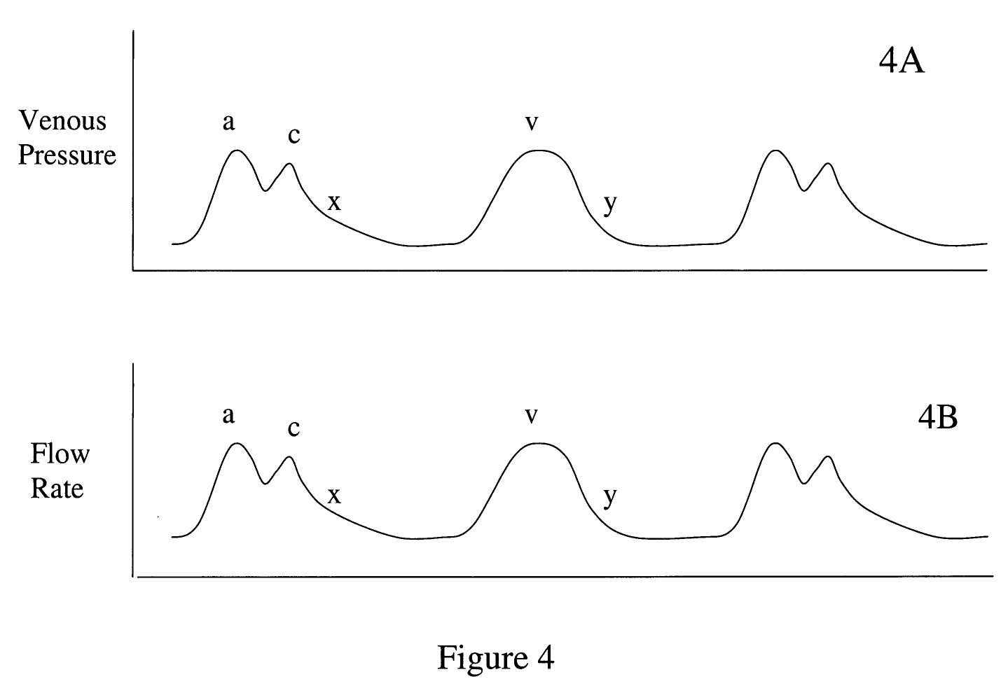

The infusion system shown in FIGS. 1 and 2 may be represented by an electrical system as shown in FIG. 3. Since the liquid reservoir is placed above the body into which the liquid is infused, the difference in height between the liquid reservoir and the body generates a pressure head. This pressure head has the electrical equivalent of a battery and is shown as P1 in FIG. 3. This pressure head is shown as a constant for purposes of illustration, although for gravity based infusion systems an essentially constant pressure head is a good approximation. In reality, as liquid is infused, the pressure head will slowly decrease since the average height of the liquid above the body decreases. The infusion set, including the flow cell, provides a fixed resistance to the flow and is represented by R1 in FIG. 3. Because administration of the fluid into the body in an IV administration system is into the vein, the rate of liquid flow as shown in FIG. 3 will vary slightly due to blood pressure variations in the vein. These blood pressure variations are illustrated in FIG. 4 as graph 4A. The flow symbols a, c, x, v, and y are those taken from the course lecture of Dr. Mark as mentioned above. Since these variations are pressure variations, they are shown as variable battery P2 in the circuit in FIG. 3. As the pressure in the infusion system changes as shown in FIG. 4A, the flow of fluid also changes; these changes, shown in FIG. 4B, are in direct proportion to the pressure changes.

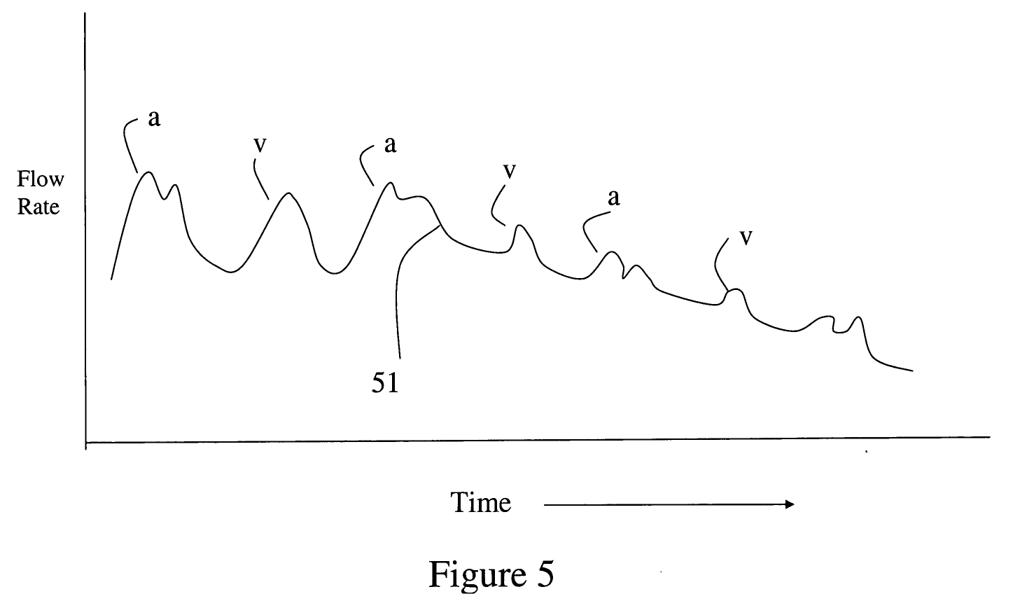

If the flow of the liquid were always into the vein, the circuit would be complete with only elements P1, R1 and P2. Unfortunately, occasionally the cannula exit port, which provides access to the body, is either not properly placed in the vein or moves to an extravascular location during the infusion. Such an instance, known as either an infiltration or an extravasation, is electrically equivalent to an additional resistance in the circuit. This additional resistance is variable and unknown. When the cannula or exit port of the IV infusion set is properly placed in the vein, this term is essentially zero and this circuit component may be ignored. However, when the cannula is not in the vein, it may constitute a large resistance, dramatically reducing flow. Alternatively, the cannula may exit the vein into a relative tissue void, significantly increasing flow. In any event of extravascular location of the cannula, the transition may be abrupt, but thereafter the change in resistance is relatively slow. FIG. 5 shows an example of one of these events when the cannula, properly placed in the vein, shows the prominent fluctuations of flow due to venous pressure variations until the cannula moves to an extravascular location, identified at time 51 in FIG. 5. After that event, the flow rate rapidly declines with much less prominent flow rate variations due to venous pressure variations. The flow rate change at a typical time point 51 may be relatively small, as shown in FIG. 5, or may be a large positive change if the cannula moves to a tissue void, or may be a large negative change if the cannula moves into relatively dense tissue.

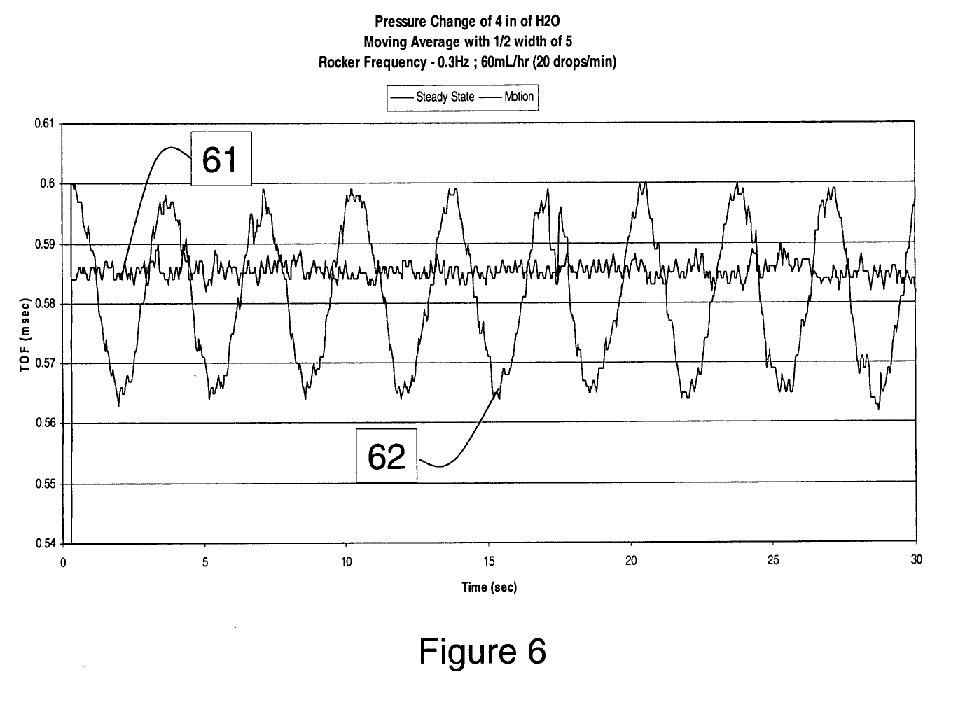

An infusion similar to the infusion system shown in FIG. 1 was set up in the laboratory. The only difference between the laboratory system and the system in FIG. 1 was that the exit port was not placed in a vein; it was placed on a rocker capable of moving the exit port vertically a variable distance with the time of one complete vertical oscillation of about three seconds. Flow from a gravity bag was established so that the fluid was exiting the exit port. With the fluid flowing, the rocker was made to move the exit port a vertical distance thereby simulating venous pressure fluctuations. The flow rate sensor used in this experiment was an optical thermal time of flight sensor as described by Sage in US patent application 20050005710, the contents of which are incorporated herein in their entirety by reference. The sensor measured the fluid flow at a rate of 30 times per second. The results of a first experiment are shown in FIG. 6. Trace 61 shows a plot of the thermal time of flight (TOF) as measured by the sensor versus time for a period of 30 second when the rocker was at rest. Trace 62 shows the thermal time of flight (TOF) as measured by the sensor when the rocker was rocking with a vertical amplitude of 4 inches. As can be seen in FIG. 6, the flow rate changes due to the change in pressure head of 4 inches are large and easily distinguishable.

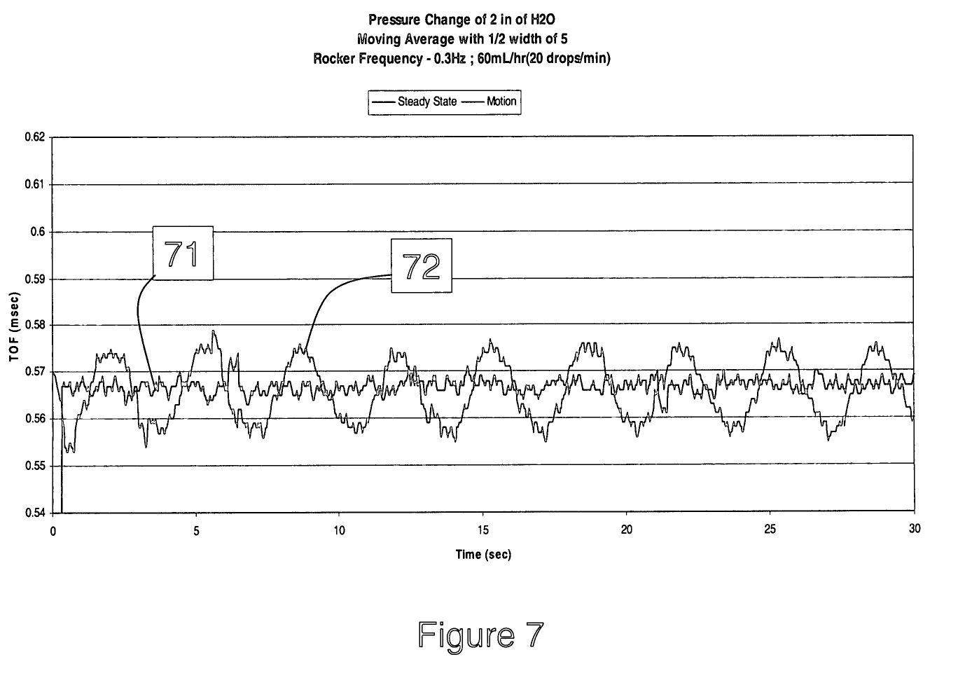

FIG. 7 shows the results of a second experiment with the experimental system described above where the vertical motion of the rocker was two inches instead of 4 inches. Otherwise, the experimental setup was identical to that used to generate the data shown in FIG. 6. Trace 71 in FIG. 7 shows the flow rate as measured by thermal time of flight (TOF) for a period of 30 seconds when the rocker was at rest. Trace 72 in FIG. 7 shows the flow rate as measured by the thermal time of flight (TOF) when the rocker moved the exit port of the infusion system a vertical distance of 2 inches. Again, the flow rate changes due to the change of pressure from the vertical motion of the exit port are prominent.

The data shown in FIGS. 6 and 7 demonstrate the sensitivity of the flow rate sensor to small changes in pressure. The vertical motion of the rocker simulates the small pressure changes that are present in a human vein. The pressure change due to a vertical change in height of the exit port of 4 inches is roughly equivalent to a pressure change of 8 mm of mercury. The pressure change due to a vertical change in height of 2 inches is roughly equal to a pressure change of 4 mm of mercury. From the data shown by Dr. Ward in his seminar on central venous pressure cited above, the amplitude of pressure changes as seen in the vein are of the same order of magnitude, that is, in the range of about 3 to 20 mm of mercury. Thus the infusion system of the invention is sensitive enough to measure flow rate changes due to venous pressure fluctuations.

The data shown in FIGS. 6 and 7 have been subjected to only a minimum of filtering to reduce noise. It is clear to those skilled in the art that a low pass filter would improve the signal to noise in the data. Additional methods to improve the signal to noise would include correlating these flow rate changes with signals generated by the heart. Such signals may be generated by a sensor placed in an artery, by using the signals from an EKG, the signals generated by a pulse oximeter, or any similar device used to monitor heart function.

We claim:

1. An IV infusion system comprising
a) a source of liquid to be infused,
b) a conduit with an exit port for conducting the liquid from the source to the patient wherein the exit port conducts the liquid into the body,
c) a sensor for measuring at least one property of liquid flow in the conduit, and
d) a processor in electrical communication with the flow sensor for processing liquid flow properties measured by the flow sensor to determine changes in flow resulting from changes in pressure at the exit port.

2. The IV infusion system of claim 1 further comprising algorithms for use by the processor for establishing that flow properties measured by the sensor are characteristic of either a properly placed exit port or an improperly placed exit port.

3. The IV infusion system of claim 2 further comprising a circuit to trigger the generation of a signal recognizable by the user of the IV system that the processor has determined that the exit port is improperly placed.

4. The IV infusion system of claim 3 wherein the flow property is one of volumetric flow rate, flow stream velocity, or pressure drops across a flow restrictor in the infusion set.

5. The IV infusion system of claim 1 wherein the processor receives electrical signals related to heart function.

6. The infusion system of claim 1 further comprising a display for displaying the changes in flow.

7. A method for properly placing the exit port of a pharmaceutical solutions delivery system in the vein of a patient comprising
a) viewing venous pressure waveforms or signals related to venous pressure waveforms, and
b) adjusting the location of the exit port such that the waveforms are prominent.

8. A method for properly placing the exit port of a pharmaceutical solutions delivery system in the vein of a patient comprising
a) employing the fluid delivery system of claim 5
b) adjusting the location of the exit port in the vein such that the displayed waveforms are prominent.
